(19) United States
(12) Patent Application Publication
(10) Pub. No.: US 2026/0295221 A1
(43) Pub. Date: Oct. 1, 2026

(54) DEVICE AND METHOD FOR LOCATING INTERNAL CAVITY TISSUES

(71) Applicant: Shenzhen Kaiyan Medical Equipment Co., Ltd., Shenzhen (CN)

(72) Inventors: Alain Dijkstra, Amstelveen (NL); Chen Shoufeng, Shenzhen (CN); Lu Zhenquan, Shenzhen (CN)

(73) Assignee: Shenzhen Kaiyan Medical Equipment Co., Ltd., Shenzhen (CN)

(21) Appl. No.: 19/576,516

(22) Filed: Mar. 24, 2026

(30) Foreign Application Priority Data
Mar. 27, 2025 (CN) 2025103703793

(51) Int. Cl. A61M 25/10

(52) U.S. Cl. CPC A61M 25/10; A61M 2025/1043

(57) ABSTRACT

The present invention discloses a device and a method for locating tissues within internal body cavities during diagnostic or surgical procedures. The device comprises a catheter assembly having a lumen formed from a light-transmissive material, an expandable balloon body disposed at a distal end of the catheter assembly for stable positioning within a body cavity, and a light-emitting component including an external controller and an optical fiber. The optical fiber includes a light-emitting section positioned near the balloon body and configured to emit light along the length of the catheter assembly to mark a surgical cutting boundary in real time. Light generated by the controller is transmitted through the optical fiber without generating heat within the body. The method comprises inserting the catheter assembly, inflating the balloon body to position the light-emitting section at a target surgical area, and transmitting light to mark the surgical cutting boundary in real time.

### TECHNICAL FIELD

The present invention relates generally to the field of medical device technology and, more particularly, to a minimally invasive device for locating, identifying, and marking tissues within internal body cavities or anatomical passages during diagnostic or surgical procedures.

### BACKGROUND ART

Conventional techniques for locating and identifying tissues within internal body cavities during surgical procedures primarily rely on visual estimation through endoscopic imaging, tactile feedback, or indirect anatomical landmarks. In procedures such as prostate surgery, ureteral intervention, or other minimally invasive operations involving narrow or complex internal cavities, surgeons often depend on endoscopes, imaging guidance, or preoperative scans to infer the location of critical tissues and surgical boundaries. In some cases, balloons or catheters are used solely for dilation, positioning, or drainage, without providing real-time visual localization of the target tissue boundary.

In prostate surgery, existing procedures reduce urethral resistance by removing the diseased and enlarged portion of the prostate. However, due to the close anatomical relationship between the prostate and the urethra, the urethra, which is surrounded by the prostate, is difficult to identify. This makes it easy to inadvertently cut into the urethra when removing the diseased prostate, resulting in the inability to urinate independently during a recovery period of approximately 3 to 5 days after surgery. Patients may require a catheter to assist with urination during this recovery period.

These traditional approaches suffer from several limitations. During many intracavitary surgical or diagnostic procedures, accurately identifying target tissues can be challenging due to limited visibility, anatomical variability, and the risk of inadvertent injury to surrounding structures. Conventional catheter-based devices may provide mechanical positioning or fluid management, but often lack integrated visual guidance mechanisms capable of clearly delineating tissue regions in real time. Visual identification through endoscopes may be obstructed by blood, turbid fluids, tissue swelling, or limited viewing angles. Anatomical variations between patients further reduce the reliability of landmark-based positioning. Imaging modalities such as ultrasound, fluoroscopy, or MRI increase procedural complexity, cost, and setup time, and may not provide continuous real-time feedback during tissue resection. Existing catheters and balloon devices lack integrated, precise, and uniform tissue-marking capabilities, making it difficult to delineate surgical boundaries clearly. As a result, there is an increased risk of inadvertent damage to adjacent healthy tissues, incomplete resection, prolonged operative time, and postoperative complications.

Prior art catheter devices have attempted to address some of these challenges through various approaches. For example, transilluminating Foley catheters have been developed to facilitate prostate and bladder surgery by incorporating optical fibers within an illumination lumen to emit light at one or more points along the length of the catheter. Such devices include an elongated flexible tube with a drainage lumen, an inflatable balloon, and an illumination adapter configured to optically couple a light source to the catheter assembly. The flexible elongated light-emitting element emits sensible light through the bladder and through the urethra to illuminate the bladder, the bladder neck, the urethra adjacent to the prostate, and surrounding tissue. To enable emission of light at determined points, cladding is partially or fully removed from the optical fiber. However, these transilluminating devices are primarily designed to differentiate opaque tissue structures from translucent structures by passing light through tissue, rather than to provide real-time marking of surgical cutting boundaries. Furthermore, such devices typically position the light-emitting elements within the bladder and urethra for general illumination purposes, rather than positioning a light-emitting section near the balloon body specifically to mark a surgical area during tissue resection. These devices also do not incorporate spectroscopic analysis of reflected light to distinguish between diseased and normal tissue, nor do they include metal sensing capabilities for detecting the proximity of surgical instruments during tissue resection.

Other prior art devices have employed balloon catheters with integrated light irradiation capabilities for phototherapy or optical therapeutic applications. In such devices, a light irradiation body may be inserted into a balloon catheter to emit light of a predetermined wavelength toward surrounding tissue for therapeutic purposes, such as photoimmunotherapy or photodynamic therapy. These optical therapeutic devices are designed to deliver therapeutic light doses to tissue rather than to provide visual localization or boundary marking for surgical guidance. The light emission in such devices is typically configured for treatment effect rather than for real-time visualization of tissue locations during resection procedures. Moreover, such devices do not analyze reflected light to characterize tissue type or detect the proximity of metallic surgical instruments.

Additionally, catheter devices incorporating optical fibers for ultraviolet sterilization have been developed, wherein optical fibers are disposed within a catheter wall and configured to emit ultraviolet light inwardly toward the catheter lumen to reduce bacterial colonization and prevent catheter-associated infections. Such sterilization catheters include protective components to prevent ultraviolet light from exiting the outer wall of the catheter, thereby directing light inward for antimicrobial purposes rather than outward for tissue visualization. These devices are not configured to emit visible light outward through the catheter assembly to mark surrounding tissues or surgical boundaries, nor do they incorporate spectroscopic measurement or metal sensing functionalities.

Further prior art includes catheter devices incorporating scintillators and optical fibers for radiation detection during brachytherapy procedures. In such devices, scintillators embedded in the catheter wall produce light in response to incident radiation from an external radioactive source, and optical fibers transmit the resulting light signals to external detection units for monitoring radiation dose and source position. These radiation detection catheters function as passive sensing devices that receive and transmit light generated by external radiation rather than as active illumination devices that emit controlled light outwardly to mark tissue locations. Such devices do not provide real-time spectroscopic analysis of tissue characteristics or proximity detection of surgical instruments.

Other prior art catheter systems have incorporated proximity sensors to detect the position and presence of surgical instruments within defined regions of the catheter. Such sensorized Foley catheters include sensor lumens extending the length of the catheter body, with sensors configured to activate visual or audible alert notification systems when instruments are detected in proximity to the urethra or other anatomical structures. While these devices provide detection warnings to reduce the risk of puncture and lessen reliance on image guidance, they do not provide active illumination for real-time visual marking of surgical boundaries. Furthermore, such devices do not integrate optical fiber-based light emission with metal sensing capabilities in a single catheter assembly, nor do they provide spectroscopic analysis of reflected light for tissue characterization.

Additionally, intelligent sensing catheters have been developed that incorporate position sensors and data-acquisition components to monitor the position of the catheter during insertion by detecting changes in the wall thickness of peripheral organs. Such devices may include light source components and photosensitive components that emit detection light and acquire reflected light intensity information to determine whether the catheter head has reached the bladder. However, these sensing catheters are designed for catheter positioning feedback rather than for marking surgical cutting boundaries during tissue resection procedures. Such devices do not provide controlled outward light emission for surgical boundary marking, spectroscopic analysis of reflected light for distinguishing diseased from normal tissue, or metal sensing for detecting the proximity of surgical instruments during tissue resection.

Accordingly, despite the existence of various catheter-based devices incorporating optical fibers for transillumination, phototherapy, sterilization, radiation sensing, proximity detection, or position monitoring, a gap remains in the art for a device that combines stable intracavitary positioning with one or more of: (a) controlled, uniform, outward-directed light emission through a light-transmissive catheter material specifically configured for real-time visual marking of surgical cutting boundaries during tissue resection; (b) spectroscopic analysis of reflected light propagating back through the optical fiber to distinguish between diseased tissue and normal tissue, thereby enabling detection of damaged tissue during a surgical procedure; and (c) metal sensing for detecting the proximity of metallic surgical instruments relative to the catheter assembly and providing real-time feedback to an operator to control surgical resection depth and reduce the risk of damage to surrounding healthy tissue. There is a need for a tissue-locating device having adjustable light parameters, a compact cross-sectional profile suitable for minimally invasive insertion, and the capability to provide continuous, circumferentially uniform illumination of a target surgical area without generating heat within the body cavity or exposing surrounding tissues to potentially harmful radiation.

The present invention addresses these challenges by providing a catheter-based tissue-locating device incorporating an expandable balloon body for stable intracavitary positioning and an optical fiber configured to emit light from within the body cavity. By delivering controlled illumination through an optical fiber extending along the catheter assembly, the device enables real-time visual marking of target tissues or surgical areas, thereby enhancing procedural accuracy and safety.

In particular, the catheter assembly of the present invention includes one or more lumens configured to accommodate fluid delivery, optical transmission, and fluid drainage in a compact and integrated structure. The light-emitting section of the optical fiber is positioned near the balloon body and extends longitudinally along the catheter assembly, allowing emitted light to uniformly illuminate surrounding tissues while maintaining a reduced device profile suitable for minimally invasive insertion.

Advantageously, the optical fiber is operably connected to an external controller capable of adjusting light intensity, wavelength, and emission characteristics based on procedural requirements, tissue type, or patient-specific factors. This configurability allows the device to be adapted for use across a range of clinical applications, including but not limited to urological, gynaecological, gastrointestinal, and other intracavitary procedures. Unlike traditional devices, the present invention separates light generation from light transmission, enabling the use of a small-diameter optical fiber and maintaining a reduced cross-sectional profile of the catheter assembly. This facilitates smooth insertion and navigation within narrow anatomical passages while avoiding thermal damage associated with internal light sources. Furthermore, the invention may incorporate a spectroscopic measurement unit configured to analyze reflected light transmitted back through the optical fiber to distinguish between diseased and normal tissue, thereby enabling precise control of surgical resection depth. The invention may also incorporate a metal sensor including a metal sensing coil configured to detect the proximity of metallic surgical instruments and provide real-time feedback to the operator, thereby reducing the risk of inadvertent damage to surrounding healthy tissue or to the catheter assembly during tissue resection. These combined features of uniform circumferential light emission, adjustable light intensity and wavelength, spectroscopic tissue analysis, and metal sensing for instrument proximity detection enhance accuracy, safety, and adaptability across different surgical applications.

### OBJECTS OF THE INVENTION

Some of the objects of the invention are as follows:

An object of the present invention is to provide a device and method for locating tissues in internal body cavities that enables clear and real-time identification of surgical regions during minimally invasive procedures.

Another object of the present invention is to provide a tissue-locating device comprising a catheter assembly having a proximal end and a distal end, wherein the distal end is insertable into an internal body cavity, while the proximal end remains externally accessible for control and connection.

Another object of the present invention is to provide a device having an optical fiber extending between an input end and an output end, wherein light introduced at the input end is transmitted through the optical fiber and emitted from the output end for illuminating and marking a target tissue region.

A further object of the present invention is to provide a tissue-locating device incorporating a light-emitting section disposed on the catheter assembly, thereby enabling illumination of tissue boundaries within an internal anatomical cavity.

Another object of the present invention is to provide a device comprising a wavelength control knob configured to adjust the wavelength/color of emitted light (red, blue, green, and combinations thereof), thereby allowing adaptation of the emitted light characteristics to different tissue types, surgical requirements, or visualization conditions.

Another object of the present invention is to provide a controller including a power adjustment knob configured to regulate the optical power delivered to the light-emitting section, thereby enabling controlled illumination intensity/brightness of the light during surgical procedures.

A further object of the present invention is to provide a catheter assembly comprising an expandable balloon body disposed near the distal end, wherein the balloon body is configured to expand within the internal cavity to stabilize the device and maintain proper positioning relative to the target tissue.

Another object of the present invention is to provide a device having a light-transmissive catheter wall or light-emitting section configured to allow light emitted from the optical fiber to radiate outwardly and visually indicate the surgical boundary.

Another object of the present invention is to provide a tissue-locating device with a compact, minimally invasive structural configuration, thereby facilitating smooth insertion, navigation, and positioning within narrow or complex anatomical passages.

A further object of the present invention is to provide a device capable of delivering uniform and continuous illumination along a predetermined length of the catheter assembly, thereby improving visualization accuracy during surgical operations.

Yet another object of the present invention is to provide a safe and reliable tissue-localization solution that assists surgeons in accurately identifying anatomical boundaries, thereby reducing the risk of inadvertent tissue injury and improving surgical precision and clinical outcomes.

### SUMMARY OF THE INVENTION

According to a first aspect of the present invention, a device for locating tissues in internal cavities is provided. The device comprising: a catheter assembly having a lumen extending along a length thereof, the catheter assembly having a first through-hole formed at a distal end thereof and in fluid communication with the lumen, the catheter assembly being formed from a light-transmissive material configured to permit light to pass outwardly therethrough; a balloon body disposed at the distal end of the catheter assembly and fluidly connected to the first through-hole, the balloon body being configured to expand upon delivery of a fluid through the lumen and the first through-hole to position the catheter assembly within an internal body cavity; and a controller arranged outside the catheter assembly and an optical fiber having an input end operably connected to the controller and an output end extending through the lumen, the output end of the optical fiber having a light-emitting section located on a side of the balloon body near the distal end of the catheter assembly, the light-emitting section extending along a longitudinal direction of the catheter assembly, wherein light generated by the controller is transmitted through the optical fiber to the light-emitting section and light emitted from the light-emitting section illuminates surrounding tissue to indicate a boundary of a surgical region.

In one embodiment of the invention, the catheter assembly comprises a plurality of lumens, including a first lumen for fluid delivery to the balloon body and a second lumen for accommodating the optical fiber.

In one embodiment of the invention, the lumen comprises a plurality of channels, wherein the plurality of channel comprises a ballon channel to allow fluid from the lumen to the balloon body, an optical path channel for transmission of the optical fiber, and a drainage channel configured to drainage of a waste fluid.

In one embodiment of the invention, the catheter assembly comprises an inner tube and an outer tube arranged concentrically.

In one embodiment of the invention, the light-emitting section is configured to emit light circumferentially around the catheter assembly.

In one embodiment of the invention, the light-emitting section comprises a diffusing region formed by modification of a cladding layer of the optical fiber, wherein the modification comprises at least one of notching, thinning, or partial removal of the cladding layer.

In one embodiment of the invention, the light-emitting section is helically arranged within the catheter assembly.

In one embodiment of the invention, the controller is configured to adjust at least one of the wavelength or intensity of the light.

In one embodiment of the invention, the balloon body is configured to stabilize the catheter assembly by radial expansion within the internal body cavity.

According to a second aspect of the invention, a device for locating tissues in internal cavities is provided. The device comprising: a catheter assembly defining a lumen and being formed from a light-transmissive material configured to permit light to pass outwardly therethrough; an optical fiber disposed within the lumen of the catheter assembly, the optical fiber having an input end configured to receive light from a light-emitting component and an output end having a light-emitting section configured to emit light toward surrounding tissue; wherein at least a portion of the emitted light is reflected from the surrounding tissue and propagates back toward the optical fiber; and a spectroscopic measurement unit operably coupled to the optical fiber and configured to receive the reflected light transmitted through the optical fiber and to analyze spectral characteristics of the reflected light, to distinguish between diseased tissue and normal tissue, thereby enabling detection of damaged tissue within the internal body cavity during a surgical procedure.

In one embodiment of the invention, the spectroscopic measurement unit is configured to analyze at least one of intensity, phase, or wavelength distribution of the reflected light.

In one embodiment of the invention, the spectroscopic measurement unit is configured to compare the spectral characteristics of the reflected light with reference spectral data to classify tissue type.

In one embodiment of the invention, the optical fiber is configured to both emit light and receive reflected light.

In one embodiment of the invention, the spectroscopic measurement unit is configured to perform real-time analysis during the surgical procedure.

In one embodiment of the invention, the spectroscopic measurement unit is configured to operate based on at least one of fluorescence spectroscopy, reflectance spectroscopy, or Raman spectroscopy.

According to a third aspect of the invention, a device for locating tissues in internal cavities is provided. The device comprising: a catheter assembly configured to be positioned within an internal body cavity; an optical fiber disposed within the catheter assembly and having a light-emitting section configured to emit light toward surrounding tissue to visually indicate a surgical boundary during a tissue resection procedure; a metal sensor including a metal sensing coil disposed adjacent to a distal portion of the catheter assembly and configured to detect proximity of a metallic surgical instrument; a signal converter configured to convert a signal generated by the metal sensing coil into a corresponding electrical detection signal; and a controller operably connected to the metal sensor and the signal converter and configured to receive the electrical detection signal corresponding to the proximity of the metallic surgical instrument and to determine a relative position of the surgical instrument with respect to the catheter assembly, wherein the controller is further configured to generate a feedback signal based on the detected proximity of the metallic surgical instrument and to provide at least one of an audible alarm, a visual indicator, or a control signal to alert an operator when the metallic surgical instrument approaches within a predetermined threshold distance from the catheter assembly, and wherein the feedback signal enables real-time adjustment of a depth or thickness of tissue resection during the surgical procedure and to reduce a risk of damage to surrounding healthy tissue or to the catheter assembly.

In one embodiment of the invention, the controller is configured to determine the relative position based on a magnitude or variation of the electrical detection signal.

In one embodiment of the invention, the predetermined threshold distance is adjustable.

In one embodiment of the invention, a spectroscopic measurement unit is operably coupled to the optical fiber and configured to receive a reflected light transmitted through the optical fiber and to analyze spectral characteristics of the reflected light, to mark the surgical boundary.

In one embodiment of the invention, the controller is configured to continuously monitor proximity in real time during the surgical procedure.

In the context of the specification, when an element is referred to as being “fixed to” or “disposed to” another element, it may either be directly on another element or indirectly on that other element. When a component is said to be “connected” or “connected to” another component, it may be directly connected to another component or indirectly connected to other components on the piece.

In the context of the specification, the terms “first”, “second,” and “third” are only used for descriptive purposes and do not imply the relative importance or implicitly indicate the quantity of technical features indicated.

In the context of the specification, the term “plurality” means two or more than two, unless otherwise indicated.

In various embodiments, the body cavity or channel may include, but is not limited to, the ureter, intestines, oesophagus, pulmonary passages, nasal cavity, blood vessels, oral cavity, or similar anatomical structures. The surgical area may include, but is not limited to, prostate, bladder, uterus, ovaries, intestines, kidneys, liver, pancreas, stomach, ears, nose, heart, lungs, or other internal body cavities. The fluid used to expand the balloon body may be a gas, saline solution, or another suitable inflation medium, without limitation.

For purposes of this application, the terms "proximal" and "distal" are defined with reference to an operator. The end of a component closer to the operator is referred to as the proximal end, and the end farther from the operator is referred to as the distal end.

In the context of the specification, the term “light-emitting component” encompasses any light-emitting device capable of emitting light of therapeutic wavelength(s), including but not limited to light-emitting diodes (LEDs), organic LEDs (OLEDs), laser diodes, or equivalent optical sources. The light may include ultraviolet, visible, near-infrared, or far-infrared spectra.

In the context of the specification, the term “housing” is intended to cover any casing, enclosure, or structural body that contains or supports components of the device. The housing may include a handle portion, head portion, or other segments, and may be made from polymeric, metallic, composite, or other suitable materials.

In the context of the specification, the term "LED” refers to one or more light-emitting diode (LED) elements that are electrically connected and configured to emit light of specific wavelengths suitable for therapeutic purposes. The LED module may include drive circuitry, heat dissipation structures, and optical elements such as lenses or diffusers to control light distribution.

In the context of the specification, the term “light source” or “phototherapy source” etc. refers to a source emitting coherent laser light, or light-emitting diodes (“LEDs”). The term “light therapy” refers to light generated from any of the sources, such as lasers, LED sources, or Super luminous diodes (“SLD”).

In the context of the specification, “Light Emitting Diodes (LEDs)” refer to semiconductor diodes capable of emitting electromagnetic radiation when supplied with an electric current. The LEDs are characterized by superior power efficiencies, smaller sizes, rapid switching speeds, physical robustness, and longer lifespans compared to incandescent or fluorescent lamps. The one or more LEDs may include through-hole type LEDs (generally emitting electromagnetic radiation in red, green, yellow, blue, and white colors), Surface Mount Technology (SMT) LEDs, Bi-color LEDs, Pulse Width Modulated RGB (Red-Green-Blue) LEDs, and high-power LEDs, among others.

Materials used in one or more LEDs may vary from one embodiment to another, depending upon the frequency of radiation required. Different frequencies can be obtained from LEDs made from pure or doped semiconductor materials. Commonly used semiconductor materials include nitrides of Silicon, Gallium, Aluminum, Boron, Zinc Selenide, etc., in pure form or doped with elements such as Aluminum and Indium. For example, red and amber colors are produced from Aluminum Indium Gallium Phosphide (AlGaInP) based compositions, while blue, green, and cyan use Indium Gallium Nitride based compositions. White light may be produced by mixing red, green, and blue lights in equal proportions, while varying proportions may be used to generate a wider color gamut. White and other colored lightings may also be produced using phosphor coatings such as Yttrium Aluminum Garnet (YAG) in combination with a blue LED to generate white light, and Magnesium-doped potassium fluorosilicate in combination with a blue LED to generate red light.

In addition to conventional mineral-based LEDs, one or more LEDs may also be provided on an Organic LED (OLED) based flexible panel or an inorganic LED-based flexible panel. Such OLED panels may be generated by depositing organic semiconducting materials over Thin Film Transistor (TFT) based substrates. Further, a discussion on the generation of OLED panels can be found in Bardsley, J. N (2004), “International OLED Technology Roadmap”, IEEE Journal of Selected Topics in Quantum Electronics, Vol. 10, No. 1, that is included herein in its entirety, by reference. An exemplary description of flexible inorganic light-emitting diode strips can be found in granted U.S. Pat. No. 7,476,557 B2, titled “Roll-to-roll fabricated light sheet and encapsulated semiconductor circuit devices”, which is included herein in its entirety by reference.

Unless otherwise stated, the term “light” as used in this specification encompasses electromagnetic radiation in the visible (380–780 nm) and infrared (780 nm–1000 nm) ranges, particularly red light (620–750 nm) and near-infrared (750–1400 nm) wavelengths commonly used in photo-biomodulation therapy. Particular wavelengths which may be selected as the dominant emissive wavelength may include the following, without any preference to be indicated by order: 400 nm, 405 nm, 420 nm, 430 nm, 450 nm, 465 nm, 515 nm, 530 nm, 532 nm, 590 nm, 630 nm, 633 nm, 640 nm, 650 nm, 655 nm, 660 nm, 670 nm, 680 nm, 780 nm, 785 nm, 810 nm, 830 nm, 840 nm, 850 nm, 860 nm, 870 nm, 904 nm, 915 nm, 980 nm, 1015 nm, 1060 nm, 1065 nm, 1070 nm, 1200 nm, and 1400 nm. As used herein, the term “light therapy” refers to the use of one or more light sources of any type that emit light with a wavelength between about 400 and 1400 nm. The device may also emit blue or ultraviolet light for surface-level treatments such as acne reduction or microbial control.

### BRIEF DESCRIPTION OF THE ACCOMPANYING DRAWINGS

The accompanying drawings illustrate the best mode for carrying out the invention as presently contemplated and set forth hereinafter. The present invention may be more clearly understood from a consideration of the following detailed description of the preferred embodiments taken in conjunction with the accompanying drawings, wherein like reference letters and numerals indicate the corresponding parts in various figures in the accompanying drawings, and in which:

FIG. 1 shows a schematic diagram of a bladder, a prostate, and a urethra of the human body, in accordance with an embodiment of the present invention.

FIG. 2 is a perspective view of a catheter device for locating internal cavity tissues, in accordance with an embodiment of the present invention.

FIG. 3 illustrates the structure of the catheter device for locating internal cavity tissues with the catheter assembly removed, in accordance with an embodiment of the present invention.

FIG. 4 is a schematic diagram of the installation of the optical fiber, in accordance with an embodiment of the present invention.

FIG. 5 is a cross-sectional view along line A-A in FIG. 2, in accordance with an embodiment of the present invention.

FIG. 6 is a partial cross-sectional view of the optical fiber in the device for locating internal cavity tissues, in accordance with an embodiment of the present invention.

FIG. 7 is a first installation schematic diagram of the catheter assembly and the optical fiber, in accordance with an embodiment of the present invention.

FIG. 8 is a second installation schematic diagram of the catheter assembly and the optical fiber, in accordance with an embodiment of the present invention.

### DETAILED DESCRIPTION

Embodiments of the present invention disclosure will be described more fully hereinafter with reference to the accompanying drawings in which like numerals represent like elements throughout the figures, and in which example embodiments are shown.

The detailed description and the accompanying drawings illustrate the specific exemplary embodiments by which the disclosure may be practiced. These embodiments are described in detail to enable those skilled in the art to practice the invention illustrated in the disclosure. It is to be understood that other embodiments may be utilized, and other changes may be made, without departing from the spirit or scope of the present disclosure. The following detailed description is therefore not to be taken in a limiting sense, and the scope of the present invention disclosure is defined by the appended claims. Embodiments of the claims may, however, be embodied in many different forms and should not be construed as limited to the embodiments set forth herein.

The terms “a” and “an” herein do not denote a limitation of quantity, but rather denote the presence of at least one of the referenced items. The terms “having”, “comprising”, “including”, and variations thereof signify the presence of a component.

Embodiments of the present invention disclose a catheter device and method for locating and positioning tissues within an internal body cavity during a medical procedure. The invention provides an integrated catheter-based system configured to emit light from within the internal body cavity to visually indicate a tissue location, boundary, or region of interest.

The catheter device includes a catheter assembly having at least one lumen extending along a longitudinal direction, a balloon body disposed at a distal end of the catheter assembly, and an optical fiber operably connected to an external controller. The balloon body is configured to receive fluid to expand and position the catheter assembly within the internal body cavity, while the optical fiber extends through the catheter assembly to deliver light to a light-emitting section positioned near the balloon body.

The light-emitting section of the optical fiber extends along the length of the catheter assembly and is configured to emit light outwardly through the catheter assembly to mark a surgical area or tissue location. The catheter assembly may include multiple mutually independent channels configured to support fluid delivery to the balloon body, optical transmission through the optical fiber, and fluid drainage within the internal body cavity.

Further, the optical fiber and the catheter assembly may be formed from light-transmissive materials and may include light-diffusing structures configured to distribute emitted light uniformly. The optical fiber is operably connected to a controller configured to regulate light output parameters, including intensity and wavelength, to suit procedural requirements or tissue characteristics.

Accordingly, the present invention provides a structured and controllable system for intracavitary tissue localization and visualization, enabling accurate identification of tissue regions during medical procedures through internal light emission and catheter-based positioning.

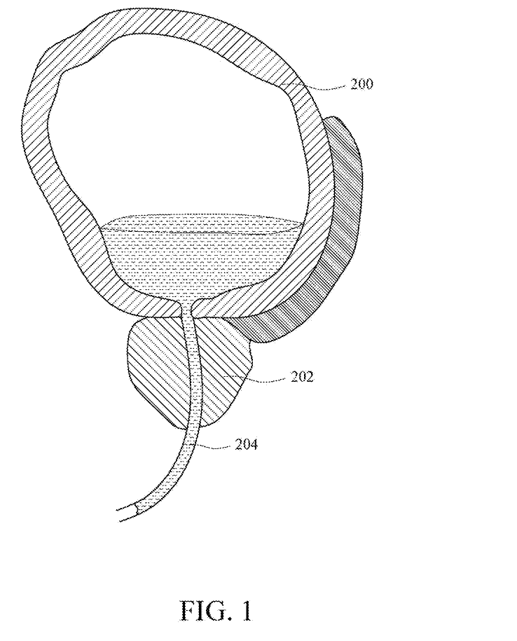

Referring to FIG. 1, a schematic diagram of a bladder 200, a prostate 202, and a urethra 204 of the human body is shown. The bladder 200 is illustrated as a rounded structure positioned at the upper portion of the figure. The prostate 202 surrounds a portion of the urethra 204, with the prostate 202 shown as a region encircling the urethra 204 below the bladder 200. The urethra 204 extends downward from the bladder 200 and passes through the prostate 202, continuing beyond the prostate 202 toward a lower portion of the figure. This anatomical arrangement illustrates the spatial relationship between the bladder 200, the prostate 202, and the urethra 204, which is relevant to understanding the positioning and operation of the catheter device of the present invention during urological procedures.

In many patients, enlargement of the prostate 202 increases urethral resistance, requiring higher intravesical pressure during voiding and contributing to bladder wall hypertrophy and increased stiffness. The combined effects of elevated urethral resistance, increased voiding pressure, and bladder wall hypertrophy can give rise to a variety of lower urinary tract symptoms (LUTS), which may significantly impair a patient's quality of life. Such lower urinary tract symptoms may include, but are not limited to, weak or intermittent urinary stream, straining during urination, hesitancy before urination, a sensation of incomplete bladder emptying following urination, post-void dribbling or leakage, increased urinary frequency, particularly nocturia, and urinary urgency.

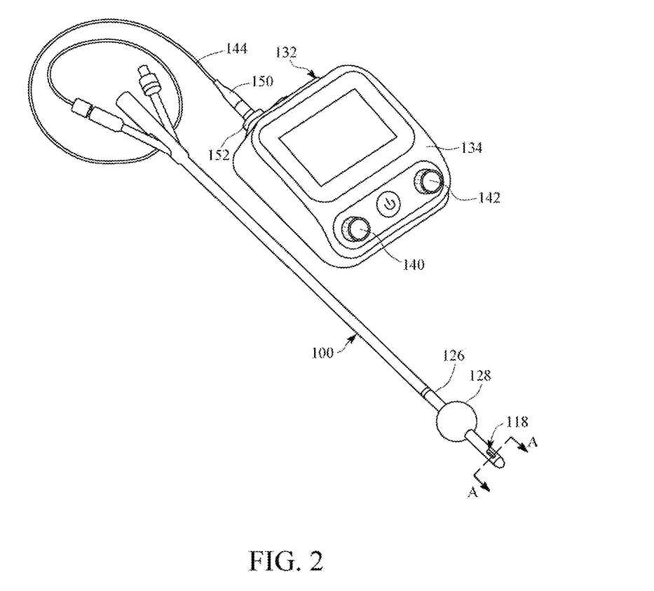

Referring to FIG. 2, a perspective view of a catheter device for locating internal cavity tissues is provided. The device comprises a catheter assembly 100 extending longitudinally from a controller 132. The catheter assembly 100 includes a light-diffusing surface 126 positioned along a portion of its length and terminates at a balloon body 128 disposed at a distal end thereof. A second through hole 118 is provided on the catheter assembly 100 adjacent to the balloon body 128. The controller 132 comprises a housing 134 that integrates a power adjustment knob 140 and a wavelength control knob 142, which are accessible on an exterior surface of the housing 134. An optical fiber 144 extends from the controller 132 into the catheter assembly 100. An input end of the optical fiber 144 is provided with a first connector 150, which detachably connects to a second connector 152 provided on the controller 132. This detachable connection between the first connector 150 and the second connector 152 enables convenient assembly and disassembly of the optical fiber 144 and the controller 132, allowing selective use of different models of controllers and optical fibers.

The first connector 150 and the second connector 152 use a standard FC optical fiber interface between the optical fiber 144 and the controller 132. The optical fiber is formed from a PMMA optical fiber serving as a light-guiding medium. The optical fiber has an outer diameter of approximately 1 mm and an inner diameter of approximately 0.75 mm.

The optical fiber 144 is a transparent or semi-transparent light guide rod, or a light-transmissive catheter wall formed from a biocompatible polymer material. The optical fiber 144 extends longitudinally along at least a portion of the catheter body, thereby enabling the delivery of the phototherapy light emitted by a light-emitting component 136 disposed at the distal end of the catheter assembly 100, and hence to an internal tissue region.

The light emitted from the optical fiber 144 acts as a medium for providing phototherapy for treating internal tissues. The phototherapeutic light may promote wound healing, tissue regeneration, inflammation reduction, and cellular repair at or adjacent to the surgical site. The phototherapy light is selected to have wavelengths and intensities that are non-destructive to biological tissue and do not cause thermal injury, necrosis, or structural damage, while instead stimulating beneficial photo-biomodulation effects to support post-resection healing and tissue recovery within the internal body cavity.

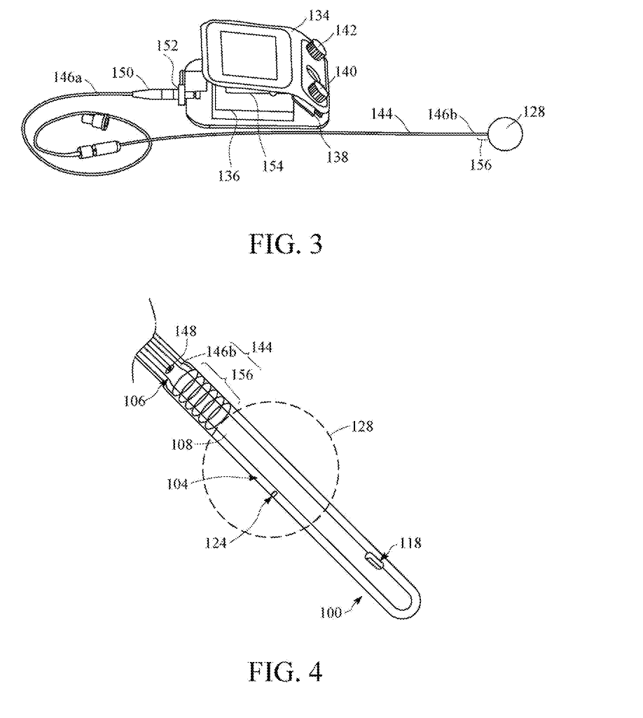

Referring to FIGS. 3 and 4, the optical fiber 144 is operably connected to the controller 132 via the first connector 150 disposed at an input end 146a, which engages with the second connector 152 provided on the housing 134 of the controller 132. An output end 146b of the optical fiber 144 aligns with a light-emitting section 156 positioned on the distal end, along the length of the catheter assembly 100. The optical fiber 144 is operably connected to the controller via the first connector 150 disposed at the input end 146a, which engages with the second connector 152 provided on the housing 134. The housing 134 of the controller 132 integrates the light-emitting component 136 and a control board 138 configured to control the operation of the light-emitting component 136. The power adjustment knob 140 is provided on the housing 134 of the controller 132, which is electrically connected to the control board 138 for adjusting optical output power and the brightness of the light emitted by the light-emitting component 136. The Wavelength control knob 142 is also provided on the housing 134 for selectively activating different wavelengths of the light-emitting component 136, which in turn emit light in different colors.

A metal sensing coil 148 is disposed on a side of the light-emitting section 156 near to the balloon body 128 and is electrically connected to a signal converter integrated within the controller 132. A spectroscopic measurement unit 154 is provided within the housing 134 and is configured to receive and analyze reflected light transmitted back through the optical fiber 144. The catheter assembly 100 includes the balloon body 128 disposed at a distal end thereof and fluidly connected to a first through-hole 124. The lumen of the catheter assembly 100 includes a balloon channel 104, an optical path channel 106 within which the optical fiber 144 is disposed, and a drainage channel 108 configured to allow fluid to flow from the distal end to the proximal end of the catheter assembly 100. The second through hole 118 is provided on a side wall of the catheter assembly 100 for drainage of body fluids. The light-emitting section 156 extends along a longitudinal direction of the catheter assembly 100 and is configured to emit light outwardly through the catheter assembly 100 to mark a surgical area in real time.

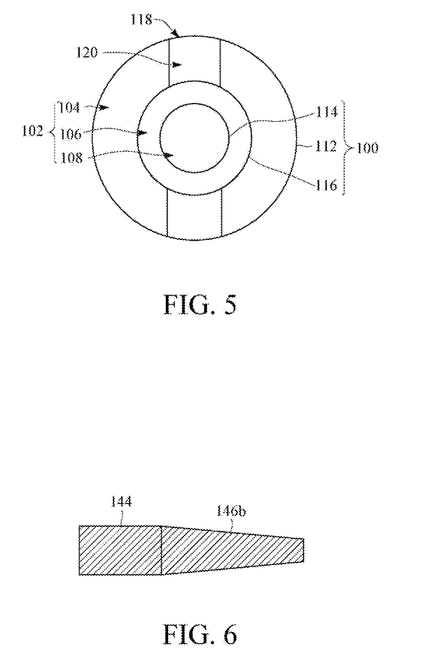

Referring to FIG. 5, the catheter assembly 100 includes a first transparent tube 112, a second transparent tube 114, and a third transparent tube 116 arranged in a concentrically nested configuration from an outer side toward an inner side. The balloon channel 104 is defined between the first transparent tube 112 and the third transparent tube 116. The drainage channel 108 is defined between the third transparent tube 116 and the second transparent tube 114. The optical path channel 106 is defined within the second transparent tube 114, and the optical fiber 144 with the light-emitting section 146 is disposed within the optical path channel 106. The second through hole 118 is provided on a side wall of the first transparent tube 112, and a liquid guide channel 120 is sealingly connected between the second through hole 118 and the drainage channel 108. The liquid guide channel 120 defines a low-resistance fluid pathway configured to facilitate rapid drainage of body fluids. The concentric arrangement of the transparent tubes provides physical isolation between the balloon channel 104, the optical path channel 106, and the drainage channel 108, enabling independent control of balloon inflation, optical transmission, and fluid drainage functions.

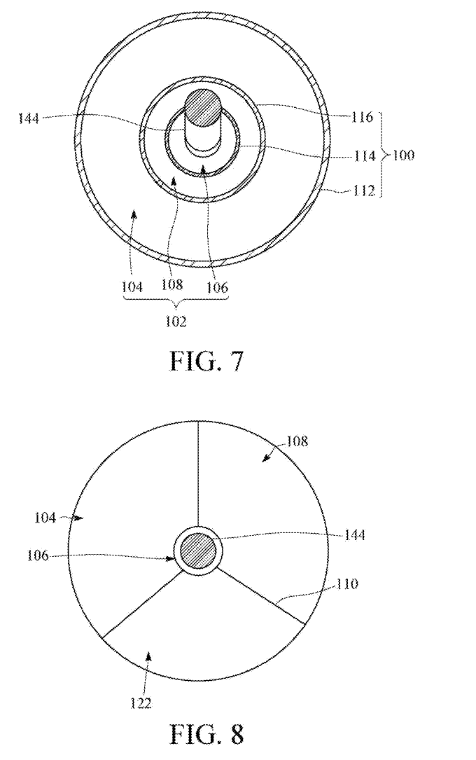

Referring to FIG. 7, the catheter assembly 100 includes the first transparent tube 112, the second transparent tube 114, and the third transparent tube 116 arranged in a concentrically nested configuration from an outer side toward an inner side. The optical path channel 106 is disposed substantially at a central region of the lumen 102, and the optical fiber 144 is positioned within the optical path channel 106 at the center of the catheter assembly 100. A partition wall 110 is formed within the lumen 102 to divide the interior space into the balloon channel 104, the optical path channel 106, the drainage channel 108, and a drug delivery channel 122. In this configuration, the optical path channel 106 is centrally located with a relatively small cross-sectional area, which facilitates central positioning of the optical fiber 144 and ensures circumferentially uniform light emission.

Referring to FIG. 8, an alternative channel arrangement within the catheter assembly 100 is provided, wherein the partition wall 110 is integrally formed within the lumen 102 to divide the interior space into the balloon channel 104, the optical path channel 106, the drainage channel 108, and the drug delivery channel 122. This configuration differs from FIG. 7 in the relative positioning and arrangement of the channels, illustrating that the shapes and relative positions of the channels may be varied while maintaining functional decoupling between fluid delivery, optical transmission, and fluid drainage functions.

Referring to FIG. 6, a partial cross-sectional view of the optical fiber 144 is shown, illustrating the light-emitting section 156 of the optical fiber 144. The figure illustrates the gradual decrease in outer diameter of the light-emitting section 156 in the direction away from the input end 146a. The light-emitting section 156 may have a conical outer surface formed by a machining or engraving process to enhance light diffusion and achieve uniform brightness along its length. This tapered configuration compensates for the gradual reduction in luminous flux along the length of the light-emitting section 156, ensuring substantially uniform light emission throughout the light-emitting section 156.

In an embodiment, the optical fiber 144 comprises a fiber core and a cladding, wherein a refractive index of the fiber core is greater than a refractive index of the cladding, such that light propagates through the fiber core by total internal reflection. The light-emitting section 156 may include only the fiber core, or may include both the fiber core and the cladding, wherein a thickness of the cladding at the light-emitting section 156 is smaller than a cladding thickness at other portions of the optical fiber 144, or wherein the cladding at the light-emitting section 156 is locally notched, thinned, or otherwise modified. As a result, light leaks from the fiber core at the light-emitting section 156 and is scattered outwardly, thereby enabling the light-emitting section 156 to emit light. The light-emitting section 156 is configured to have a small outer diameter so as not to increase the overall outer diameter or cross-sectional area of the catheter assembly 100.

In an embodiment, the length of the light-emitting section 156 is in a range of approximately 30 mm to 50 mm, thereby accommodating anatomical differences among different patient populations, including adults and children. For example, the light-emitting section 156, having a length of approximately 30mm may be used for pediatric patients to achieve short-distance, high-density light scattering suitable for relatively narrow body cavities. In contrast, the light-emitting section 156, having a length of approximately 50 mm, may be used for adult patients to achieve longer-distance, lower-density light scattering, thereby covering larger lesion regions, such as benign prostatic hyperplasia with a prostate volume greater than approximately 80mL.

Referring to FIGS. 2 to 4, the first through-hole 124 is formed at the distal end of the catheter assembly 100 and is in fluid communication with the lumen 102. The balloon body 128 is disposed at the distal end of the catheter assembly 100 and is fluidly connected to the first through-hole 124, such that fluid delivered through the lumen 102 can enter the balloon body 128.

The controller 132 and the optical fiber 144 are arranged outside the catheter assembly 100. The input end 146a of the optical fiber 144 is operably connected to the controller 132, while the output end 146b of the optical fiber 144 is provided with the light-emitting section 156. The light-emitting section 156 is located on a side of the balloon body 128 near the distal end of the catheter assembly 100 and extends substantially along a longitudinal direction of the catheter assembly 100. Light generated by the light-emitting component 136 of the controller 132 is transmitted through the optical fiber 144 to the light-emitting section 156 and is emitted outward through the catheter assembly 100.

In operation, the distal end of the catheter assembly 100 is inserted into a human body through a body cavity or channel. A fluid is delivered through the lumen 102 and the first through-hole 124 into the balloon body 128, thereby causing the balloon body 128 to expand. Expansion of the balloon body 128 enables the catheter assembly 100 and the balloon body 128 to be collectively positioned and stably retained within the body cavity or channel, thereby achieving accurate positioning relative to a surgical area. In this positioned state, the light-emitting section 156 is aligned with the target surgical area.

Light emitted by the light-emitting component 136 is transmitted through the optical fiber 144 to the light-emitting section 156 and exits through the catheter assembly 100, thereby marking the location of the body cavity or channel within the surgical field. In this manner, the device provides a real-time indication of a surgical cutting boundary, effectively addressing the technical problem associated with difficulty in identifying surgical areas in existing procedures. As a result, the risk of inadvertent tissue damage or miscutting caused by complex anatomical structures is significantly reduced.

Furthermore, since the controller 132 is positioned outside the catheter assembly 100 and the optical fiber 144 serves only as a light-transmission medium without generating light internally, the optical fiber 144 has a small outer diameter and minimal space requirements. Consequently, the catheter assembly 100 can be designed with a reduced cross-sectional area, thereby facilitating smooth insertion and navigation within the human body.

In an embodiment, the body cavity or channel may include, but is not limited to, the ureter, intestines, oesophagus, pulmonary passages, nasal cavity, blood vessels, oral cavity, or similar anatomical structures. The surgical area may include, but is not limited to, the prostate, bladder, uterus, ovaries, intestines, kidneys, liver, pancreas, stomach, ears, nose, heart, lungs, or other internal body cavities. The fluid used to expand the balloon body 128 can be a gas, saline solution, or other suitable inflation media, without limitation.

For purposes of this application, the terms “proximal” and “distal” are defined with reference to an operator. The end of a component closer to the operator is referred to as the proximal end, and the end farther from the operator is referred to as the distal end.

In an embodiment, the optical fiber 144 being fixedly positioned within the lumen 102 means that at least a portion of the optical fiber 144 is secured within the lumen 102 of the catheter assembly 100. The optical fiber 144 may be integrally formed with the catheter assembly 100, such as by co-molding or overmolding, to achieve fixed positioning. Alternatively, the optical fiber 144 may be manufactured separately from the catheter assembly 100 and subsequently fixed within the catheter assembly 100 by bonding, welding, snap-fitting, interference fitting, or other suitable fixation methods.

In an embodiment, the device further comprises the spectroscopic measurement unit 154 configured to cooperate with the optical fiber 144 to detect and analyze light reflected from tissue within the body cavity. The controller 132 drives the light-emitting component 136 to emit light, which is coupled into the input end of the optical fiber 144. The optical fiber 144 transmits the light along its length through total internal reflection within the fiber core, thereby minimizing transmission loss and maintaining signal integrity. The transmitted light reaches the light-emitting section 156, where structural modification of the cladding permits controlled leakage of light, such that the light is emitted radially outward into the surrounding tissue region.

In use, after the catheter assembly 100 is positioned within the body cavity and the light-emitting section 156 is aligned with a target tissue region, the emitted light propagates into the adjacent biological tissue. Upon interaction with the tissue, a portion of the incident light is absorbed, while another portion is scattered and reflected due to variations in tissue composition, density, and morphology. The reflected component, referred to as backscattered or back-reflected light, carries information indicative of the optical properties of the tissue, including absorption characteristics, scattering coefficients, and structural heterogeneity.

The back-reflected light is subsequently recoupled into the optical fiber 144 through the same light-emitting section 156. Due to the bidirectional transmission capability of the optical fiber, the reflected light propagates toward the input end of the optical fiber 144 and is directed to the spectroscopic measurement unit 154. The spectroscopic measurement unit 154 receives the reflected optical signal and performs spectral analysis thereon, including measurement of intensity attenuation and phase delay of the returned light signal, as described in the specification.

In particular, the intensity attenuation corresponds to the reduction in amplitude of the reflected light relative to the emitted light, which is influenced by the absorption characteristics of the tissue. Tissues with higher absorption, such as diseased or damaged tissues exhibiting abnormal vascularization or composition, result in greater attenuation of specific wavelengths. Conversely, normal tissues exhibit relatively consistent and predictable attenuation profiles. By comparing the detected intensity profile with reference or baseline values, the spectroscopic measurement unit 154 is able to distinguish between normal and abnormal tissue regions.

Additionally, the phase delay of the reflected light is indicative of changes in optical path length caused by variations in tissue refractive index and structural organization. Damaged or pathological tissues typically exhibit altered microstructure, leading to differences in light propagation speed and scattering behaviour. These differences manifest as measurable phase shifts in the returned signal. The spectroscopic measurement unit 154 processes the phase information to further characterize the tissue and enhance discrimination accuracy.

The controller 132 may sequentially activate different wavelengths of light from the light-emitting component 136, and the spectroscopic measurement unit 154 may analyze the wavelength-dependent response of the reflected light. Since different tissue types exhibit distinct spectral signatures across multiple wavelengths, this multispectral analysis enables more precise identification of damaged tissue regions based on their unique optical response patterns.

Accordingly, by integrating controlled light emission, bidirectional optical fiber transmission, and spectroscopic analysis of reflected light, the device enables real-time detection and differentiation of tissue conditions within a body cavity. The coordinated analysis of intensity attenuation and phase delay allows the system to accurately identify damaged or abnormal tissue regions, thereby assisting in the precise localization of surgical targets and reducing the risk of inadvertent damage to healthy tissue.

In an embodiment, with reference to FIGS. 6 to 8, the cross-sectional shape of the catheter assembly 100 and the cross-sectional shape of the light-emitting section 156 may be independently selected from circular, elliptical, polygonal, or irregular geometries, without limitation. The term “cross-section” refers to a section taken perpendicular to the longitudinal direction of the catheter assembly 100. The outer diameter of the catheter assembly 100 refers to the diameter of a circumscribed circle enclosing the cross-section thereof, and the outer diameter of the light-emitting section 156 similarly refers to the diameter of a circumscribed circle enclosing its cross-section.

In an embodiment, the optical fiber 144 includes a fiber core and a cladding, wherein a refractive index of the fiber core is greater than a refractive index of the cladding, such that light propagates through the fiber core by total internal reflection. The light-emitting section 156 may include only the fiber core, or may include both the fiber core and the cladding, wherein a thickness of the cladding at the light-emitting section 156 is smaller than a cladding thickness at other portions of the optical fiber 144, or wherein the cladding at the light-emitting section 156 is locally notched, thinned, or otherwise modified. As a result, light leaks from the fiber core at the light-emitting section 156 and is scattered outwardly, thereby enabling the light-emitting section 156 to emit light. The light-emitting section 156 is configured to have a small outer diameter so as not to increase the overall outer diameter or cross-sectional area of the catheter assembly 100.

In an embodiment, with reference to FIGS. 3 and 6, the outer diameter of the light-emitting section 156 gradually decreases near the output end 146b, in a direction away from the input end 146a of the optical fiber 144. During light propagation, a portion of light leaks from the light-emitting section 156, resulting in a gradual reduction in luminous flux along the length of the light-emitting section 156, which would otherwise cause non-uniform brightness. By gradually reducing the outer diameter of the light-emitting section 156, the contact area between the guided light and a cladding or an external medium is increased, such that the proportion of light leakage from the light-emitting section 156 progressively increases along the length thereof. In this manner, the increased light leakage dynamically compensates for the decrease in luminous flux, thereby achieving substantially uniform brightness along the length of the light-emitting section 156 and avoiding misidentification of surgical boundary locations during a surgical procedure.

Specifically, referring to FIG. 6, the light-emitting section 156 can be formed using a machining or engraving process to provide the gradually decreasing outer diameter. For example, a conical engraving process may be applied to the surface of the light-emitting section 156 such that an outer surface thereof has a conical profile, with the outer diameter decreasing smoothly and continuously in the direction away from the input end 146a of the optical fiber 144. As a result, the proportion of light leaking from the light-emitting section 156 increases in a corresponding smooth and continuous manner, and, in combination with the gradual reduction in luminous flux, uniform light emission along the entire length of the light-emitting section 156 is achieved.

Referring to FIG. 2, in an embodiment, an outer circumferential surface of the light-emitting section 156 is provided with the light-diffusing surface 126 configured to uniformly disperse emitted light, and/or an outer circumferential surface of the catheter assembly 100 is provided with the light-diffusing surface 126. In this manner, substantially 360-degree circumferentially uniform light emission is achieved, enabling clear visualization and accurate positioning of surgical area boundaries from multiple viewing angles during a surgical procedure. This configuration improves procedural accuracy and safety, reduces the risk of miscutting caused by complex anatomical structures or insufficient illumination, and eliminates the need to rotate the optical fiber 144 to adjust a light emission direction.

In an embodiment, the light-diffusing surface 126 can include approximately 50 to 150 micro-protrusions per millimeter, thereby enhancing light scattering uniformity.

In an embodiment, the outer circumferential surface of the light-emitting section 156 and/or the catheter assembly 100 is coated with a quantum dot material, such as CdSe/ZnS quantum dots. The quantum dot material is configured to convert incident light, such as red light, into light having a wavelength in a range of approximately 610nm to 630nm, thereby enhancing tissue penetration depth and improving visualization and positioning performance within biological tissues.

In an embodiment, the light-diffusing surface 126 may be formed as a frosted surface or as a scattering particle coating, thereby increasing optical interfaces and improving light diffusion performance. In other embodiments, the light-diffusing surface 126 may be formed on the outer circumferential surface of the light-emitting section 156 and/or the catheter assembly 100 using a laser etching process. For example, a micron-scale concave-convex array may be engraved on the outer circumferential surface of the light-emitting section 156 to disrupt conditions for total internal reflection and induce multi-angle light scattering.

In an embodiment, an outer circumferential surface of the light-emitting section 156 and/or the catheter assembly 100 is coated with a titanium dioxide nanoparticle-doped resin. Titanium dioxide has a relatively high refractive index and is capable of efficiently scattering incident light, thereby enhancing light diffusion. In addition, nano-scale titanium dioxide particles exhibit high surface activity and interact with molecules in the resin matrix to form strong chemical bonds, thereby improving coating adhesion and preventing detachment, peeling, or degradation of the coating during use.

In an embodiment, a length of the light-emitting section 156 is in a range of approximately 30 mm to 50 mm, thereby accommodating anatomical differences among different patient populations, including adults and children. For example, the light-emitting section 156, having a length of approximately 30 mm, may be used for pediatric patients to achieve short-distance, high-density light scattering suitable for relatively narrow body cavities. In contrast, the light-emitting section 156, having a length of approximately 50 mm, may be used for adult patients to achieve longer-distance, lower-density light scattering, thereby covering larger lesion regions, such as benign prostatic hyperplasia with a prostate volume greater than approximately 80mL.

In an embodiment, with reference to FIGS. 4, 7, and 8, the lumen 102 includes a plurality of mutually independent channels, including the balloon channel 104, the optical path channel 106, and the drainage channel 108. A distal end of the balloon channel 104 is fluidly connected to the first through-hole 124, and the optical fiber 144 is disposed within the optical path channel 106. The drainage channel 108 extends from the distal end to the proximal end of the catheter assembly 100 and is configured exclusively for draining internal body fluids. For example, when the device is applied in prostate surgery, the drainage channel 108 may be used for draining urine from the bladder.

In an embodiment, by physically isolating the drainage channel 108 for fluid drainage, the balloon channel 104 for controlling inflation and deflation of the balloon body 128, and the optical path channel 106 for optical transmission of the optical fiber 144, functional decoupling and synergistic optimization are achieved. On the one hand, this configuration enables independent and precise control of the expansion and contraction of the balloon body 128 and allows selection of the balloon channel 104 having an appropriate inner diameter to improve balloon expansion efficiency. On the other hand, the optical path channel 106 can be configured with an inner diameter smaller than that of the balloon channel 104, thereby facilitating stable positioning of the light-emitting section 156 of the optical fiber 144 and reducing fluctuations in emitted light intensity.

Referring to FIG. 4, the optical path channel 106 is arranged in an annular configuration around the circumference of the lumen 102, and a light-emitting section 156 is helically or spirally disposed within the optical path channel 106. In this helical configuration, the spiral annular light-emitting section 156 improves the circumferential uniformity of light emission and reduces the standard deviation of the circumferential light intensity distribution. In addition, the spiral arrangement increases the illuminated area, reduces the occurrence of dark spots or localized bright spots, and increases the effective light-emitting length by approximately three to five times relative to a linear configuration. Moreover, positioning the light-emitting section 156 closer to an outer circumference of the catheter assembly 100 shortens the optical transmission path, thereby further improving emitted brightness. Additionally, the spiral configuration enhances the compressive strength of both the catheter assembly 100 and the light-emitting section 156, reducing displacement of the optical path caused by friction or mechanical interaction with surgical instruments during endoscopic procedures.

In an embodiment, with reference to FIG. 7, in a cross-sectional view of the catheter assembly 100, the optical path channel 106 is disposed substantially at a central region of the lumen 102, and the light-emitting section 156 is configured as a linear structure extending along a longitudinal direction of the optical path channel 106. In this configuration, the light-emitting section 156 is positioned at a central axis of the catheter assembly 100, such that light is emitted radially outward from the center toward surrounding tissues, thereby avoiding unilateral overexposure caused by an eccentric light field. Furthermore, the linear light-emitting section 156 has a small outer diameter, which is advantageous for miniaturization of the catheter assembly 100.

In an embodiment, the distal end of the optical path channel 106 is sealed, thereby isolating the light-emitting section 156 from micro-vibrations or disturbances caused by fluid flow within adjacent channels.

Referring to FIG. 8, in an embodiment, the partition wall 110 is integrally formed within the lumen 102 to divide an interior space thereof into the balloon channel 104, the optical path channel 106, and the drainage channel 108. The partition wall 110 thereby provides a seamless internal structure without interface stress concentration points. This configuration eliminates the need for additional assembly space or connecting structures and facilitates an ultra-fine outer diameter design of the catheter assembly 100. Furthermore, the shapes and relative positions of the balloon channel 104, the optical path channel 106, and the drainage channel 108 are fixed by the integrally formed partition wall 110, thereby minimizing or eliminating leakage risks between channels.

In an embodiment, the catheter assembly 100 includes the first transparent tube 112, the third transparent tube 116, and the second transparent tube 114 arranged in a concentrically nested configuration from an outer side toward an inner side. The balloon channel 104 is defined between the first transparent tube 112 and the third transparent tube 116, the drainage channel 108 is defined between the third transparent tube 116 and the second transparent tube 114, and the optical path channel 106 is defined within the second transparent tube 114. In this configuration, the optical path channel 106 is centrally located and has a relatively small cross-sectional area, which facilitates central positioning of the optical fiber 144, ensures circumferentially uniform light emission, and maintains optical signal integrity. The balloon channel 104 and the drainage channel 108 are annular channels having relatively large cross-sectional areas, thereby facilitating uniform and rapid fluid flow.

Additionally, the first transparent tube 112, the third transparent tube 116, and the second transparent tube 114 may each be formed from transparent silicone material. Silicone materials exhibit visible-light transmittance in a range of approximately 85% to 99%, excellent biocompatibility, and a bending fatigue life of up to approximately 100,000 cycles, thereby facilitating smooth advancement of the catheter assembly 100 through blood vessels, the urethra, or other body passages.

At least one of the first transparent tube 112, the third transparent tube 116, and the second transparent tube 114 is provided with the light-diffusing surface 126, such as a frosted or micro-textured structure, at a region corresponding to the light-emitting section 156, thereby improving uniformity of emitted light transmitted through the catheter assembly 100.

In an embodiment, the balloon body 128 may be integrally formed with the catheter assembly 100, thereby providing a seamless connection between the catheter assembly 100 and the balloon body 128. Such an integral structure avoids interface stress concentration points, eliminates the need for additional assembly space and connecting structures, and facilitates a reduced cross-sectional profile of the balloon body 128. In other embodiments, the balloon body 128 may be formed separately from the catheter assembly 100 and subsequently connected thereto by bonding, welding, snap-fitting, interference fitting, or other suitable connection techniques.

In an embodiment, the optical fiber 144 being fixedly positioned within the lumen 102 means that at least a portion of the optical fiber 144 is secured within the lumen 102 of the catheter assembly 100. The optical fiber 144 may be integrally formed with the catheter assembly 100, such as by co-molding or overmolding, to achieve fixed positioning. Alternatively, the optical fiber 144 may be manufactured separately from the catheter assembly 100 and subsequently fixed within the catheter assembly 100 by bonding, welding, snap-fitting, interference fitting, or other suitable fixation methods.

Referring to FIG. 5, in an embodiment, a side wall of the first transparent tube 112 is provided with the second through hole 118, and the lumen 102 further includes the liquid guide channel 120 that is sealingly connected between the second through hole 118 and the drainage channel 108. The liquid guide channel 120 defines a low-resistance fluid pathway, thereby facilitating rapid drainage of body fluids. In addition, the second through hole 118 is positioned on a distal end of the first transparent tube 112 rather than at an end thereof, such that the proximal end rigidity of the catheter assembly 100 is maintained, thereby improving the advancement and maneuverability of the catheter assembly 100 within blood vessels, the urethra, or similar anatomical passages.

In an embodiment, with reference to FIG. 8, the lumen 102 further includes the drug delivery channel 122. The drug delivery channel 122 is physically isolated from the drainage channel 108 used for fluid drainage, the balloon channel 104 used for controlling inflation and deflation of the balloon body 128, and the optical path channel 106 used for optical transmission of the optical fiber 144. Such physical isolation prevents cross-contamination between delivered drugs and bodily fluids, inflation media, or optical components, thereby improving safety and reliability during clinical use.

In some embodiments, the catheter assembly 100 is integrally injection-molded onto the optical fiber 144, thereby eliminating the need for separate connecting structures. This configuration facilitates a reduction in the overall outer diameter of the catheter assembly 100 and avoids issues associated with mismatched coefficients of thermal expansion that may arise in conventional adhesive bonding processes, thereby reducing temperature-induced positional drift. As a result, the optical fiber 144 is securely and precisely positioned within the catheter assembly 100.

In an embodiment, the integral injection-molding of the catheter assembly 100 onto the optical fiber 144 avoids issues associated with mismatched coefficients of thermal expansion that may arise in conventional adhesive bonding processes, thereby reducing temperature-induced positional drift. As a result, the optical fiber 144 is securely and precisely positioned within the catheter assembly 100 throughout a range of operating temperatures.

Referring to FIGS. 2 and 3, in an embodiment, the input end 146a of the optical fiber 144 extends out from the proximal end of the catheter assembly 100 and is provided with the first connector 150. The controller 132 is provided with the second connector 152, which is detachably connected to the first connector 150. In this manner, the optical fiber 144 and the controller 132 may be conveniently assembled and disassembled, enabling the selective use of different models of the controller 132 and different models of optical fiber 144, thereby improving the versatility and compatibility of the system.

In an embodiment, the controller 132 is configured to control the light-emitting section 156 to emit laser light, thereby enabling precise control of a surgical resection depth or thickness and reducing the risk of damage to surrounding normal tissue. For example, benign prostatic hyperplasia (BPH) tissue exhibits variations in internal microstructure, including differences in acinar size, stratified epithelial-like glandular arrangements, and irregular proliferation of stromal cells. Such cellular proliferation and disordered tissue architecture cause partial absorption, refraction, and attenuation of incident light as it propagates through BPH tissue, resulting in changes in optical path characteristics. Laser light, having high coherence, exhibits reduced brightness after passing through BPH tissue due to such refraction and attenuation. Once the hyperplastic tissue has been removed, the laser light passes through normal prostate tissue with reduced attenuation, thereby exhibiting increased brightness. This contrast in emitted brightness provides a clear optical indication of completion of the surgical resection and helps prevent excessive tissue removal that could otherwise result in urethral injury.

The laser source is configured to emit red light having a wavelength in the range of 650-660nm. The optical output power is approximately 20mW/cm² to ensure effective illumination of tissue. The selected wavelength and power density provide controlled penetration while maintaining patient safety.

In an embodiment, the device further comprises a built-in lithium battery configured with a low-power consumption design to enhance operational efficiency. The power management architecture of the device enables continuous operation for at least approximately 50 hours on a single full charge, thereby allowing extended use without frequent recharging.

In an embodiment, the device is configured such that the built-in battery is fully chargeable within approximately one hour, and the device is operable either in an offline battery-powered mode or in a plugged-in mode, wherein the external power cord is connected through an aviation-type plug to ensure a secure and reliable electrical connection.

In an embodiment, the display is provided on the front face of the housing 134 for displaying operating parameters real-time. The LCD screen is configured to provide visual feedback to an operator, including operational status, light intensity settings, wavelength selection, battery level, flashing frequency, system status, and other relevant parameters during a surgical procedure. A battery level indicator allows the operator to easily monitor remaining power during use. The display is not limited to an LCD screen and may comprise any suitable visual display means, including but not limited to an LED screen, OLED display, TFT display, segmented digital display, touch-sensitive display panel, or any other equivalent visual interface configured to present operational parameters to the operator. The selection of the display type may vary depending on design requirements, power consumption considerations, visibility conditions, and user interface preferences, without departing from the scope of the present invention.

The device is provided with an integrated buzzer configured to generate audible alerts. The buzzer is activated when the battery level is low. The buzzer also provides an alarm in the event of a device malfunction to promptly notify the operator.

In an embodiment, the controller 132 further includes the power adjustment knob 140 electrically connected to the control board 138. The power adjustment knob 140 is configured to adjust the optical output power of the light-emitting component 136, thereby regulating the brightness of the light generated by the light-emitting component 136. In this manner, the light emission brightness of the light-emitting section 156 can be flexibly adjusted according to different patient anatomies, tissue thicknesses, or surgical requirements, enabling accurate marking of a surgical area.

The power adjustment knob 140, a laser on/off switch, a brightness adjustment knob, and a laser frequency adjustment knob. The brightness is adjustable across ten discrete levels for precise intensity control. The frequency adjustment knob provides tactile damping at each level and is adjustable from 0 to 10Hz, with 0-1Hz adjustable in 0.1Hz steps and 1-10Hz adjustable in 1Hz steps.

In an embodiment, the light-emitting component 136 includes a plurality of multi-color LED beads, and the controller 132 further includes the Wavelength control knob 142 electrically connected to the control board 138. The Wavelength control knob 142 is configured to selectively activate or deactivate individual LED beads, such that the light-emitting component 136 generates light of different wavelengths or colors. Different wavelengths exhibit different tissue penetration characteristics, and the control board 138 may be configured to automatically or manually switch wavelengths based on tissue type and tissue thickness encountered during a surgical procedure, thereby optimizing marking brightness and visibility. For example, during angiographic procedures, the Wavelength control knob 142 may control the light-emitting component 136 to emit green light; for marking deeper tissues, the light-emitting component 136 may be controlled to emit red light; for pediatric patients with relatively thin tissues, blue-green light in a wavelength range of approximately 450 nm to 550 nm, having a penetration depth of approximately 1 mm to 2 mm, may be selected to reduce a risk of injury to deeper sensitive tissues; and for adult patients with relatively thicker tissues, near-infrared light in a wavelength range of approximately 800 nm to 1500 nm, having a penetration depth of approximately 5 mm to 10 mm, may be selected to enhance visualization of deeper lesions.

Specifically, the light-emitting component 136 integrates a six-color high-density LED and/or laser chip array, supporting light emission over a spectral range of approximately 470 nm to 1550 nm. The light-emitting component 136 cooperates with the Wavelength control knob 142 to achieve millisecond-level wavelength switching, thereby enabling rapid adaptation of emitted light characteristics during surgical procedures.

In an embodiment with reference to FIGS. 3 and 4, the device for positioning tissue within a body cavity further includes a metal sensor. The metal sensor comprises the metal sensing coil 148 and a signal converter. The signal converter is integrated within the controller 132, while the metal sensing coil 148 is disposed at a location near the balloon body 128 and on a side of the light-emitting section 156. The metal sensing coil 148 is electrically connected to the signal converter by a conductive wire. In this configuration, the metal sensing coil 148 is designed with a small outer diameter to facilitate miniaturization of the catheter assembly 100, while the relatively larger signal converter is accommodated within the controller 132. In addition, the metal sensing coil 148 is configured to detect the proximity of metallic surgical instruments in real time, thereby facilitating precise control of surgical resection thickness and reducing the risk of damage to surrounding normal tissue.

Additionally, the metal sensing coil 148 has a diameter of approximately 1.5mm and operates at an adjustable frequency in a range of approximately 1MHz to 10MHz. The metal sensing coil 148 can detect ferromagnetic or paramagnetic materials, such as iron-, nickel-, or cobalt-based alloys, within a detection range of approximately 5 mm, with a sensitivity of approximately 0.1 mm³. The signal converter integrated within the controller 132 has a response time of less than approximately 0.1ms and is configured to convert electromagnetic signals detected by the metal sensing coil 148 into digital positioning coordinates with an accuracy of approximately ±50μm. In some embodiments, an ultra-fine conductive wire, such as a silver nanowire having a diameter of approximately 20μm and an impedance of less than approximately 0.1Ω, is used to electrically connect the metal sensing coil 148 to the signal converter, thereby providing tensile resistance and resistance to electromagnetic interference and ensuring signal stability during surgical operation.

Upon identification of the damaged or abnormal tissue region based on the spectroscopic analysis performed by the spectroscopic measurement unit 154, the controller 132 is configured to generate a corresponding control signal indicative of the location and extent of the detected tissue abnormality. In response to this determination, the device facilitates positioning or activation of the metal sensing coil 148 in proximity to the identified damaged tissue region.

In an embodiment, the metal sensing coil 148 is arranged adjacent to or integrated with the catheter assembly 100 such that it may be accurately guided toward the detected tissue region. The controller 132, based on the feedback received from the spectroscopic measurement unit 154, enables precise localization by correlating the optical detection data with the positional information of the optical fiber 144. Once the damaged tissue is localized, the metal-sensing coil 148 is positioned near the target region to detect metallic surgical tools or markers, thereby ensuring proper alignment and safe execution of the surgical procedure.

Subsequently, a surgical process is initiated, which may include tissue ablation, resection, or other therapeutic intervention. The controller 132 continuously monitors signals from both the spectroscopic measurement unit 154 and the metal sensing coil 148 during the procedure. The spectroscopic measurement unit 154 provides real-time updates regarding changes in tissue optical properties, thereby enabling assessment of treatment progress, while the metal sensing coil 148 ensures that surgical instruments remain within the intended operational zone.

In an embodiment, the device further comprises an alarm generation module operatively connected to the controller 132. The alarm generation module is configured to produce an audible, visual, or haptic alert when predetermined threshold conditions are met. Such conditions may include, but are not limited to, detection of excessive proximity between a surgical instrument and healthy tissue, deviation of the instrument from the target region, abnormal spectral feedback indicating unintended tissue damage, or detection of metallic objects outside a permissible range. Upon the occurrence of any such condition, the controller 132 triggers the alarm generation module to immediately notify the operator, thereby enhancing procedural safety.

Further, the device operates in a closed-loop feedback configuration. In particular, the light emitted through the optical fiber 144, the reflected light received and analyzed by the spectroscopic measurement unit 154, and the positional or proximity data obtained from the metal sensing coil 148 collectively form a continuous feedback loop. The controller 132 processes this real-time data to dynamically adjust operational parameters, such as light intensity, emission duration, or positioning guidance of the catheter assembly 100. As the surgical process progresses, changes in the reflected light characteristics are continuously monitored to determine whether the damaged tissue has been adequately treated or removed.

Accordingly, the feedback loop enables iterative refinement of the procedure, wherein detection, positioning, monitoring, and control occur in a synchronized and continuous manner. This closed-loop operation ensures that the surgical intervention remains confined to the targeted damaged tissue region while minimizing the risk of collateral damage to surrounding healthy tissue, thereby improving precision, safety, and overall treatment efficacy.

In an embodiment, with reference to FIGS. 2 and 3, the device for positioning intracavitary tissue further includes the spectroscopic measurement unit 154. Light emitted from the light-emitting section 156 is reflected by tissue or an obstruction to form reflected light, which is received by the light-emitting section 156 and transmitted through the optical fiber 144 to the spectroscopic measurement unit 154. The spectroscopic measurement unit 154 is configured to analyze phase delay and intensity attenuation characteristics of the reflected light. In this manner, by utilizing the bidirectional light transmission capability of the optical fiber 144, optical signals may be recovered and analyzed to determine whether the light has propagated through diseased tissue or normal tissue, thereby facilitating precise control of surgical resection thickness and reducing the risk of damage to surrounding healthy tissue.

In an embodiment, the catheter assembly 100, the balloon body 128, and the optical fiber 144 are compatible with standard sterilization methods, including ethylene oxide sterilization, gamma radiation sterilization, or steam autoclave sterilization. The device may be configured as a single-use disposable device or as a reusable device compatible with repeated sterilization cycles, depending on clinical requirements and regulatory considerations.

In an embodiment, a method for locating tissues in an internal body cavity during a surgical procedure is provided. The method comprises inserting the distal end of the catheter assembly 100 into the internal body cavity, wherein the catheter assembly 100 has the lumen 102 extending along a length thereof and the optical fiber 144 disposed within the lumen 102. A fluid is delivered through the lumen 102 and the first through-hole 124 at the distal end of the catheter assembly 100 into the balloon body 128 disposed at the distal end, thereby causing the balloon body 128 to expand and position the catheter assembly 100 within the internal body cavity such that the light-emitting section 156 of the optical fiber 144 is aligned with a target surgical area.

Light is transmitted from the controller 132 arranged outside the catheter assembly 100 through the optical fiber 144 to the light-emitting section 156. The light-emitting section 156 is located near the balloon body 128 and extends along a longitudinal direction of the catheter assembly 100. The light is emitted outwardly through the catheter assembly 100 to mark a location of a surgical cutting boundary within the internal body cavity in real time, thereby enabling an operator to clearly identify the boundary between target tissue and surrounding healthy tissue during the surgical procedure.

In an embodiment, the method further comprises adjusting at least one of a light intensity and a wavelength of the light transmitted through the optical fiber 144 based on a tissue type or tissue thickness encountered during the surgical procedure. For example, adjusting the wavelength may comprise selectively activating one or more LED beads of the light-emitting component 136 within the controller 132 to emit light in a wavelength range of approximately 450 nm to 550 nm having a penetration depth of approximately 1 mm to 2 mm for pediatric patients, or approximately 800 nm to 1500 nm having a penetration depth of approximately 5 mm to 10 mm for adult patients.

In an embodiment, the method further comprises receiving reflected light at the light-emitting section 156 and transmitting the reflected light through the optical fiber 144 to the spectroscopic measurement unit 154. The spectroscopic measurement unit 154 analyzes phase delay and intensity attenuation characteristics of the reflected light to determine whether the light has propagated through diseased tissue or normal tissue, thereby facilitating precise control of surgical resection depth.

In an embodiment, the method further comprises detecting the proximity of a metallic surgical instrument using the metal sensing coil 148 disposed on a side of the light-emitting section 156 near the balloon body 128 and controlling the surgical resection thickness based on the detected proximity. This enables the operator to maintain a safe distance from the catheter assembly 100 during tissue resection and reduces the risk of inadvertent damage to the urethra or other surrounding structures.

In an embodiment, upon completion of the surgical procedure, the balloon body 128 is deflated by withdrawing fluid from the balloon body 128 through the first through-hole 124 and the lumen 102, and the catheter assembly 100 is withdrawn from the internal body cavity.

In an embodiment, the controller 132 is configured to operate the light-emitting component 136 in a pulsed or flashing light emission mode. In the pulsed mode, the light-emitting section 156 emits light in discrete pulses at a predetermined frequency, such as in a range of approximately 0.5 Hz to 20 Hz. The pulsed light emission mode may enhance visibility of the surgical cutting boundary by creating a dynamic visual contrast against surrounding tissues, thereby improving the operator's ability to distinguish the marked boundary from adjacent anatomical structures. The pulsed mode may also reduce overall light exposure to surrounding tissues and conserve the power consumption of the controller 132. In some embodiments, the operator may select between continuous and pulsed light emission modes via the control board 138 or a user interface on the housing 134. The pulsed frequency may be adjustable to accommodate different surgical requirements or operator preferences. For example, a lower frequency in a range of approximately 0.5 Hz to 2 Hz may be selected for slow, deliberate tissue resection procedures, while a higher frequency in a range of approximately 5 Hz to 20 Hz may be selected for rapid identification of tissue boundaries during exploratory procedures.

In an embodiment, the catheter assembly 100 further includes a temperature sensor disposed at or near the light-emitting section 156. The temperature sensor is configured to monitor the temperature of the light-emitting section 156 and/or surrounding tissue in real time. Temperature data acquired by the temperature sensor is transmitted to the controller 132, which may be configured to automatically reduce optical output power or deactivate the light-emitting component 136 if a detected temperature exceeds a predetermined safety threshold, such as approximately 42° C. This temperature monitoring feature provides an additional safety mechanism to prevent thermal injury to surrounding tissues during prolonged light emission. In some embodiments, the temperature sensor comprises a thermocouple, a thermistor, or a fiber Bragg grating sensor integrated within or adjacent to the optical fiber 144. The temperature sensor may be electrically connected to the controller 132 via a conductive wire extending through the lumen 102, or may transmit temperature data wirelessly to the controller 132. The controller 132 can display real-time temperature readings on the LCD screen provided on the housing 134, thereby enabling the operator to monitor tissue temperature throughout the surgical procedure. In some embodiments, the controller 132 is configured to generate an audible or visual alert when the detected temperature approaches the predetermined safety threshold, such as when the temperature exceeds approximately 38° C, thereby providing advanced warning to the operator before the safety threshold is reached.

In an embodiment, the controller 132 includes a wireless communication module configured to establish wireless connectivity with an external device, such as a smartphone, tablet, computer, or hospital information system. The wireless communication module can be operated using Bluetooth, Wi-Fi, near-field communication (NFC), or other suitable wireless communication protocols. Through the wireless communication module, the controller 132 may transmit operational data, including light intensity settings, wavelength selection, emission duration, temperature readings, spectroscopic analysis results, and metal sensing coil detection data, to the external device for real-time monitoring, data logging, and post-operative analysis. The external device may also be configured to remotely control certain functions of the controller 132, such as adjusting light intensity or wavelength, subject to appropriate safety interlocks. In some embodiments, the wireless communication module is configured to transmit data to a cloud-based storage system, enabling long-term data retention and analysis across multiple surgical procedures. The wireless communication module may also enable software updates to be wirelessly transmitted to the controller 132, thereby allowing firmware upgrades and feature enhancements without requiring physical access to the controller 132. In some embodiments, the wireless communication module is configured to communicate with other surgical instruments or monitoring devices within the operating room, enabling coordinated operation and data sharing among multiple devices during a surgical procedure.

In an embodiment, an outer surface of the catheter assembly 100 is provided with an antimicrobial coating configured to inhibit bacterial colonization and reduce the risk of catheter-associated infections. The antimicrobial coating may comprise silver nanoparticles, chlorhexidine, or other antimicrobial agents embedded in or coated onto the outer surface of the first transparent tube 112. The antimicrobial coating may be configured to provide sustained antimicrobial activity over the duration of the surgical procedure and any subsequent indwelling period. In some embodiments, the antimicrobial coating is applied to the balloon body 128 in addition to or instead of the catheter assembly 100. The antimicrobial coating may be applied using techniques such as dip coating, spray coating, or plasma deposition. In some embodiments, the antimicrobial coating comprises a combination of silver nanoparticles having a particle size in a range of approximately 10 nm to 100 nm and a biocompatible polymer matrix such as polyurethane or silicone. The silver nanoparticles may be present in a concentration of approximately 0.1% to 5% by weight relative to the polymer matrix. In some embodiments, the antimicrobial coating further includes an antibiotic agent such as rifampicin or minocycline configured to provide broad-spectrum antimicrobial activity against both gram-positive and gram-negative bacteria. The antimicrobial coating may reduce the incidence of catheter-associated urinary tract infections (CAUTIs) by approximately 50% compared to uncoated catheters.

In an embodiment, the controller 132 is configured to automatically adjust light intensity based on feedback received from the spectroscopic measurement unit 154 or other sensing components. For example, if the spectroscopic measurement unit 154 detects that reflected light intensity is below a predetermined threshold, indicating that emitted light is being absorbed or scattered by intervening tissue, the control board 138 may automatically increase the optical output power of the light-emitting component 136 to maintain adequate visibility of the surgical cutting boundary. Conversely, if reflected light intensity exceeds a predetermined threshold, indicating proximity to a tissue surface or completion of tissue resection, the control board 138 may automatically reduce optical output power to prevent overexposure. This automatic intensity adjustment feature enables adaptive illumination that responds to changing tissue conditions during the surgical procedure. In some embodiments, the controller 132 employs a proportional-integral-derivative (PID) control algorithm to continuously adjust light intensity based on real-time feedback from the spectroscopic measurement unit 154, thereby maintaining a target reflected light intensity within a predetermined range. The automatic intensity adjustment feature may be selectively enabled or disabled by the operator via the control board 138 or a user interface on the housing 134. In some embodiments, the controller 132 is configured to learn optimal light intensity settings for different tissue types based on historical data from previous surgical procedures, and to automatically apply these learned settings when similar tissue conditions are detected during a current procedure.

In an embodiment, the spectroscopic measurement unit 154 is configured to compare the spectral characteristics of the reflected light with predetermined reference spectral profiles stored in a memory component within the controller 132. The predetermined reference spectral profiles correspond to known spectral signatures of normal tissue and diseased tissue, respectively. By comparing the measured spectral characteristics, including phase delay and intensity attenuation, against the stored reference spectral profiles, the spectroscopic measurement unit 154 can determine whether the reflected light has propagated through damaged or diseased tissue. The predetermined reference spectral profiles may be established through pre-clinical calibration using tissue samples of known pathological status, and may be updated or refined based on accumulated clinical data.

In an embodiment, the spectroscopic measurement unit 154 is further configured to generate a real-time diagnostic signal based on the analyzed spectral characteristics of the reflected light. The real-time diagnostic signal may be transmitted to the controller 132, which is configured to provide at least one of a visual indication on the LCD screen or an audible alert via the integrated buzzer to assist the operator in identifying damaged tissue during the surgical procedure. For example, the LCD screen may display a color-coded indicator, such as a green indicator when the reflected light characteristics correspond to normal tissue and a red indicator when the reflected light characteristics correspond to diseased or damaged tissue. The audible alert may comprise a tone or series of tones that vary in frequency or intensity based on the degree of tissue damage detected.

In an embodiment, the device further includes a foot pedal control interface operably connected to the controller 132. The foot pedal control interface is configured to enable hands-free activation and deactivation of the light-emitting component 136 during the surgical procedure. The foot pedal control interface may include a first pedal for toggling light emission on and off, a second pedal for adjusting light intensity, and/or a third pedal for switching between different wavelength settings. The foot pedal control interface enables the operator to control light emission without removing hands from surgical instruments, thereby improving procedural efficiency and maintaining sterile technique. In some embodiments, the foot pedal control interface is connected to the controller 132 via a wired connection, such as a USB cable or proprietary connector. In other embodiments, the foot pedal control interface communicates wirelessly with the controller 132 via Bluetooth or other wireless communication protocols. The foot pedal control interface may include tactile feedback mechanisms, such as audible clicks or haptic vibrations, to confirm activation of each pedal. In some embodiments, the foot pedal control interface includes a safety interlock feature that requires simultaneous activation of two pedals to enable light emission, thereby preventing accidental activation. The foot pedal control interface may be configured to be compatible with standard surgical foot pedal systems used in operating rooms, enabling integration with existing surgical equipment.

In an embodiment, the controller 132 is configured to receive the electrical detection signal from the signal converter corresponding to the proximity of the metallic surgical instrument and to determine a relative position of the surgical instrument with respect to the catheter assembly 100. The controller 132 is further configured to generate a feedback signal based on the detected proximity of the metallic surgical instrument and to provide at least one of an audible alarm via the integrated buzzer, a visual indicator on the LCD screen, or a control signal to alert the operator when the metallic surgical instrument approaches within a predetermined threshold distance from the catheter assembly 100. The predetermined threshold distance may be set in a range of approximately 1 mm to 5 mm and may be adjustable by the operator via the control board 138. The feedback signal enables real-time adjustment of the depth or thickness of tissue resection to assist in identifying affected tissue during the surgical procedure and to reduce the risk of damage to surrounding healthy tissue or to the catheter assembly 100. In some embodiments, the controller 132 is further configured to continuously monitor a signal strength generated by the metal sensing coil 148 and to determine the proximity of the metallic surgical instrument based on variations in the signal strength. As the metallic surgical instrument approaches the catheter assembly 100, the signal strength detected by the metal sensing coil 148 increases, and as the instrument moves away, the signal strength decreases. The controller 132 may be configured to correlate the signal strength variations with distance measurements using a predetermined calibration curve stored in a memory component within the controller 132.

In an embodiment, the controller 132 is further configured to continuously monitor a signal strength generated by the metal sensing coil 148 and to determine the proximity of the metallic surgical instrument based on variations in the signal strength. As the metallic surgical instrument approaches the catheter assembly 100, the signal strength detected by the metal sensing coil 148 increases, and as the instrument moves away, the signal strength decreases. The controller 132 may be configured to correlate the signal strength variations with distance measurements using a predetermined calibration curve stored in a memory component within the controller 132.

In an embodiment, the controller 132 is further configured to continuously monitor a signal strength generated by the metal sensing coil 148 and to determine the proximity of the metallic surgical instrument based on variations in the signal strength. As the metallic surgical instrument approaches the catheter assembly 100, the signal strength detected by the metal sensing coil 148 increases, and as the instrument moves away, the signal strength decreases. The controller 132 may be configured to correlate the signal strength variations with distance measurements using a predetermined calibration curve stored in a memory component within the controller 132.

In an embodiment, the visual indicator comprises at least one of an LED indicator mounted on the housing 134 or the LCD screen configured to provide real-time visual feedback corresponding to the detected proximity of the metallic surgical instrument relative to the catheter assembly 100. The LED indicator may comprise a multi-color LED that changes color based on the detected proximity, such as green when the instrument is at a safe distance, yellow when the instrument is approaching the threshold distance, and red when the instrument is within the predetermined threshold distance. Alternatively, or additionally, the LCD screen may display a graphical proximity indicator, such as a distance bar or numerical distance readout, that updates in real time as the metallic surgical instrument moves relative to the catheter assembly 100.

In an embodiment, the controller 132 is further configured to automatically adjust at least one operational parameter of the device, including light emission from the light-emitting section 156 or a warning signal intensity, when the metallic surgical instrument is detected within the predetermined threshold distance. For example, the controller 132 may automatically increase the brightness of the light-emitting section 156 or switch to a pulsed emission mode when the metallic surgical instrument is detected in close proximity, thereby enhancing the visibility of the surgical cutting boundary and alerting the operator to exercise caution during tissue resection.

In an embodiment, the catheter assembly 100 further includes one or more radiopaque markers disposed at predetermined positions along the length thereof. The radiopaque markers are configured to be visible under fluoroscopic or X-ray imaging, thereby enabling the operator to verify the position of the catheter assembly 100 and the light-emitting section 156 relative to anatomical landmarks during the surgical procedure. The radiopaque markers may be disposed at the distal end of the catheter assembly 100, at the balloon body 128, at the light-emitting section 156, and/or at the distal end of the catheter assembly 100. The radiopaque markers may comprise barium sulfate, bismuth, tungsten, platinum, gold, or other radiopaque materials embedded in or coated onto the catheter assembly 100. In some embodiments, the radiopaque markers comprise annular bands having a width of approximately 0.5 mm to 2 mm and spaced at intervals of approximately 10 mm to 50 mm along the length of the catheter assembly 100. The radiopaque markers may be integrally molded with the catheter assembly 100 during manufacturing or may be applied as separate components after molding. In some embodiments, the radiopaque markers are configured to provide quantitative distance measurements, enabling the operator to determine the depth of insertion of the catheter assembly 100 based on the number of visible markers under fluoroscopic imaging.

In an embodiment, the controller 132 is configured to record and store operational data during the surgical procedure. The recorded data may include timestamps, light intensity settings, wavelength selections, emission durations, temperature readings, spectroscopic analysis results, metal sensing coil detection events, and user inputs. The recorded data may be stored in a memory component within the controller 132 and subsequently downloaded to an external device via the wireless communication module or a wired connection for post-operative analysis, quality control, and documentation purposes. In some embodiments, the controller 132 is configured to generate a procedural report summarizing the recorded data for inclusion in the patient's medical record. The procedural report may include graphical representations of light intensity over time, temperature profiles, spectroscopic analysis results, and annotations indicating key events during the surgical procedure. The recorded data may be encrypted to ensure patient privacy and data security. In some embodiments, the controller 132 includes a removable memory card or USB storage device for convenient data transfer. The recorded data may be analyzed using machine learning algorithms to identify patterns or trends that correlate with successful surgical outcomes, thereby enabling continuous improvement of surgical techniques and device performance.

In an embodiment, the device includes a voice control interface operably connected to the controller 132. The voice control interface includes a microphone and a voice recognition module configured to receive and interpret voice commands from the operator. The operator may use voice commands to activate or deactivate light emission, adjust light intensity, switch wavelength settings, or request status information, thereby enabling hands-free control of the device during the surgical procedure. The voice control interface may be configured to recognize a predefined set of voice commands and may include speaker verification to prevent unauthorized control. In some embodiments, the voice recognition module employs natural language processing algorithms to interpret complex voice commands and provide conversational feedback to the operator. The voice control interface may be configured to operate in noisy operating room environments by employing noise cancellation algorithms and directional microphones. In some embodiments, the voice control interface is configured to provide audible confirmation of received commands, such as by stating "light intensity increased to 50%" or "wavelength switched to 660 nanometers." The voice control interface may support multiple languages to accommodate operators from different regions. In some embodiments, the voice control interface includes a wake word or activation phrase, such as "catheter control," that must be spoken before voice commands are recognized, thereby preventing accidental activation by ambient conversation in the operating room.

In an embodiment, the catheter assembly is designed in accordance with Class III medical device aesthetics and clinical usage practices, featuring a clean medical-grade appearance and an ergonomic layout that facilitates intuitive operation and reliable use in clinical environments.

The present invention has industrial applicability in the field of medical device manufacturing and clinical instrumentation. The device may be manufactured using established catheter fabrication, optical fiber integration, and electronic control techniques, and may be produced at a commercial scale for distribution to healthcare facilities. The invention applies to a wide range of medical procedures involving internal body cavities, including but not limited to urological, gynecological, and gastrointestinal applications, thereby supporting broad commercial and clinical use.

Various modifications to these embodiments are apparent to those skilled in the art from the description and the accompanying drawings. The principles associated with the various embodiments described herein may be applied to other embodiments. Therefore, the description is not intended to be limited to the embodiments shown along with the accompanying drawings but is to provide the broadest scope consistent with the principles and the novel and inventive features disclosed or suggested herein. Accordingly, the invention is anticipated to hold on to all other such alternatives, modifications, and variations that fall within the scope of the present invention and appended claims.

1. A device for locating tissues in internal cavities, comprising:
a catheter assembly having a lumen extending along a length thereof, the catheter assembly having a first through-hole formed at a distal end thereof and in fluid communication with the lumen, the catheter assembly being formed from a light-transmissive material configured to permit light to pass outwardly therethrough;
a balloon body disposed at the distal end of the catheter assembly and fluidly connected to the first through-hole, the balloon body being configured to expand upon delivery of a fluid through the lumen and the first through-hole to position the catheter assembly within an internal body cavity; and
a controller arranged outside the catheter assembly and an optical fiber having an input end operably connected to the controller and an output end extending through the lumen, the output end of the optical fiber having a light-emitting section located on a side of the balloon body near the distal end of the catheter assembly, the light-emitting section extending along a longitudinal direction of the catheter assembly, wherein light generated by the controller is transmitted through the optical fiber to the light-emitting section and light emitted from the light-emitting section illuminates surrounding tissue to indicate the boundary of a surgical region.

2. The device as claimed in claim 1, wherein the catheter assembly comprises a plurality of lumens, including a first lumen for fluid delivery to the balloon body and a second lumen for accommodating the optical fiber.

3. The device as claimed in claim 1, wherein the lumen comprises a plurality of channels, wherein the plurality of channel comprises a ballon channel to allow fluid from the lumen to the balloon body, an optical path channel for transmission of the optical fiber, and a drainage channel configured to drainage of a waste fluid.

4. The device as claimed in claim 1, wherein the catheter assembly comprises an inner tube and an outer tube arranged concentrically.

5. The device as claimed in claim 1, wherein the light-emitting section is configured to emit light circumferentially around the catheter assembly.

6. The device as claimed in claim 1, wherein the light-emitting section comprises a diffusing region formed by modification of a cladding layer of the optical fiber, wherein the modification comprises at least one of notching, thinning, or partial removal of the cladding layer.

7. The device as claimed in claim 1, wherein the light-emitting section is helically arranged within the catheter assembly.

8. The device as claimed in claim 1, wherein the controller is configured to adjust at least one of the wavelength or intensity of the light.

9. The device as claimed in claim 1, wherein the balloon body is configured to stabilize the catheter assembly by radial expansion within the internal body cavity.

10. A device for locating tissues in internal cavities, comprising:
a catheter assembly defining a lumen and being formed from a light-transmissive material configured to permit light to pass outwardly therethrough;
an optical fiber disposed within the lumen of the catheter assembly, the optical fiber having an input end configured to receive light from a light-emitting component and an output end having a light-emitting section configured to emit light toward surrounding tissue;
wherein at least a portion of the emitted light is reflected from the surrounding tissue and propagates back toward the optical fiber; and
a spectroscopic measurement unit operably coupled to the optical fiber and configured to receive the reflected light transmitted through the optical fiber and to analyze spectral characteristics of the reflected light, to distinguish between diseased tissue and normal tissue, thereby enabling detection of damaged tissue within the internal body cavity during a surgical procedure.

11. The device as claimed in claim 10, wherein the spectroscopic measurement unit is configured to analyze at least one of intensity, phase, or wavelength distribution of the reflected light.

12. The device as claimed in claim 10, wherein the spectroscopic measurement unit is configured to compare the spectral characteristics of the reflected light with reference spectral data to classify tissue type.

13. The device as claimed in claim 10, wherein the optical fiber is configured to both emit light and receive reflected light.

14. The device as claimed in claim 10, wherein the spectroscopic measurement unit is configured to perform real-time analysis during the surgical procedure.

15. The device as claimed in claim 10, wherein the spectroscopic measurement unit is configured to operate based on at least one of fluorescence spectroscopy, reflectance spectroscopy, or Raman spectroscopy.

16. A device for locating tissues in internal cavities, comprising
a catheter assembly configured to be positioned within an internal body cavity; an optical fiber disposed within the catheter assembly and having a light-emitting section configured to emit light toward surrounding tissue to visually indicate a surgical boundary during a tissue resection procedure;
a metal sensor including a metal sensing coil disposed adjacent to a distal portion of the catheter assembly and configured to detect proximity of a metallic surgical instrument;
a signal converter configured to convert a signal generated by the metal sensing coil into a corresponding electrical detection signal; and
a controller operably connected to the metal sensor and the signal converter and configured to receive the electrical detection signal corresponding to the proximity of the metallic surgical instrument and to determine a relative position of the surgical instrument with respect to the catheter assembly,
wherein the controller is further configured to generate a feedback signal based on the detected proximity of the metallic surgical instrument and to provide at least one of an audible alarm, a visual indicator, or a control signal to alert an operator when the metallic surgical instrument approaches within a predetermined threshold distance from the catheter assembly, and
wherein the feedback signal enables real-time adjustment of the depth or thickness of tissue resection during the surgical procedure and to reduce the risk of damage to surrounding healthy tissue or to the catheter assembly.

17. The device as claimed in claim 16, wherein the controller is configured to determine the relative position based on a magnitude or variation of the electrical detection signal.

18. The device as claimed in claim 16, wherein the predetermined threshold distance is adjustable.

19. The device as claimed in claim 16, wherein a spectroscopic measurement unit is operably coupled to the optical fiber and configured to receive a reflected light transmitted through the optical fiber and to analyze spectral characteristics of the reflected light, to mark the surgical boundary.

20. The device as claimed in claim 16, wherein the controller is configured to continuously monitor proximity in real time during the surgical procedure.
